(19) United States
(12) Patent Application Publication
(10) Pub. No.: US 2008/0243011 A1
(43) Pub. Date: Oct. 2, 2008

(54) BLOOD PRESSURE MONITOR

(75) Inventor: Fumihisa HORI, Iwate-gun (JP)

Correspondence Address: SUGHRUE MION, PLLC, 2100 PENNSYLVANIA AVENUE, N.W., SUITE 800, WASHINGTON, DC, 20037, US

(73) Assignee: CITIZEN HOLDINGS CO., LTD., Tokyo (JP)

(21) Appl. No.: 12/059,192

(22) Filed: Mar. 31, 2008

(30) Foreign Application Priority Data
Mar. 30, 2007 (JP) 2007-093010

(51) Int. Cl. A61B 5/02

(52) U.S. Cl. 600/499; 600/490; 600/494

(57) ABSTRACT

The blood pressure monitor 100 includes plural case members 111 to 116 that are rotatably connected with one another to form a housing unit housing a cuff. The plural case members 111 to 116 are connected with plural torque hinges 140 or plural rotation hinges 150, and rotate to be transformed between a closed state in which the case members 111 to 116 are collapsed with the cuff housed herein, and an open state in which the case members 111 to 116 are opened.

### BACKGROUND OF THE INVENTION

1. Field of the Invention

The present invention relates to a digital blood pressure monitor that is applied to a subject to measure blood pressure, and more particularly to a blood pressure monitor that can be transformed between an open state in which a cuff is opened, and a closed state in which the cuff is enclosed.

2. Description of the Related Art

Recently, digital blood pressure monitors that automatically inflate by manipulation of a button to measure a blood pressure by a cuff wrapped around a subject (for example, an upper arm) are prevalent. Generally, in such digital blood pressure monitors, a hollow opening for inserting the arm is provided at a predetermined position of a main case, and a cuff for compressing and fixing the upper arm on an inner surface of the hollow opening is provided. Upon depression of a measurement start button, the cuff is inflated with the arm inserted in the hollow opening thereof to initiate blood pressure measurement.

To enhance storage of the digital blood pressure monitor when not in use, a technique of housing a cuff in a case has been disclosed. Specifically, a technique has been proposed in which a case cover and a cuff with plural rigid boards provided on an outer circumference thereof are connected with a link bar, the cuff is opened in a cylindrical shape when the case cover is opened, and the cuff is folded when the case cover is closed (see, for example, Japanese Patent Application Laid-open Publication No. 2002-102181).

Another technique of a finger blood pressure monitor has been proposed in which the finger blood pressure monitor includes a cover that can be opened and closed with respect to a main body and a finger cuff partially fixed on the inner side of the cover, in which the finger cuff is housed in a finger-cuff housing unit by closing the cover and into which a finger can be inserted by opening the cover (see, for example, Japanese Patent No. 3218919). Specifically, a circular spring board is circularly provided between the cuff and a curler so that by the action of the outward force constantly applied to the finger cuff, the finger cuff assumes a substantially cylindrical shape when the cover is opened. Additionally, a display for displaying a measurement result is provided on the cover.

However, the technique disclosed in the former patent document has a problem in that the display and the main body are provided on the side of the cuff, making the case larger. The technique disclosed in the latter patent document has a problem in that since the display is provided on the cover and becomes perpendicular to the finger-cuff housing unit upon measurement, a user must tilt his/her head to check a measurement result, which is troublesome. Even in the technique disclosed in the latter patent document, a problem arises in that since the finger cuff is housed in a housing case as the finger-cuff housing unit, the size of the device increases by the size of the housing case, and particularly when the device is applied to an arm blood-pressure monitor, the device becomes significantly larger.

To solve the above problems in the conventional techniques explained above, reduced device size is an object of the present invention, in particular, provision of a blood pressure monitor that can realize a reduction in size when the cuff is in a stowed state and that has enhanced display visibility.

### SUMMARY OF THE INVENTION

To solve the above problems and achieve an object, a blood pressure monitor according to the invention of claim 1, houses a cuff therein and in which at least four case members are rotatably connected with one another in a ring-shape to form a housing unit that houses the cuff, and the case members rotate to be transformed between a closed state in which the case members are collapsed with the cuff enclosed therein, and an open state.

A blood pressure monitor according to the invention of claim 2, based on the invention of claim 1, is characterized by at least one of the case members including a display.

The blood pressure monitor according to claim 3, based on the invention of claim 1, is characterized by an elastic body that exerts a force in a direction that opens the cover and is provided on the main body.

A blood pressure monitor according to claim 4, based on the invention of claim 3, is characterized by an adjusting mechanism that adjusts the force exerted by the elastic body.

The blood pressure monitor according to claim 5, based on the invention of claim 1, is characterized by the cuff including a cuff ring, an blood-flow restricting bag outside the cuff ring, and a cuff band outside the blood-flow restricting bag.

A blood pressure monitor according to claim 6, based on the invention of claim 5, is characterized by the cuff band having a circumferential extension rate lower than an axial extension rate.

A blood pressure monitor according to claim 7, based on the invention of claim 5, is characterized by the cuff ring including two openings through which support members are respectively provided, and the support members being fitted to the main body and/or the cover.

A blood pressure monitor according to claim 8, based on the invention of claim 1, further includes a first locking mechanism that includes a first locking member that is provided on one of the case members that are adjacent in a state in which the cover is open, and a first hook that is provided on the other of the case members that are adjacent in the state in which the cover is open, and at which the first locking member locks, in which the first locking mechanism maintains the state in which the cover is open.

A blood pressure monitor according to claim 9, based on the invention of claim 8, is characterized by the first locking mechanism including a first button that unlocks the first locking mechanism.

A blood pressure monitor according to claim 10, based on the invention of claim 1, further includes a second locking mechanism that includes a second locking member that is provided on one of the case members that oppose each other in a state in which the cover is closed, a second hook that is provided on the other of the case members that oppose each other in the state in which the cover is closed, and in which the second locking mechanism maintains the state in which the cover is closed.

A blood pressure monitor according to claim 11, based on the invention of claim 10, is characterized by the second locking mechanism including a second button that connects to and unlocks the second locking member.

A blood pressure monitor according to the invention of claim 12 includes a cuff; a plurality of case members that surround an outer circumference of the cuff; a plurality of hinges that rotatably connects the case members with one another to transform the case members between a open state in which the cuff is open and a stowed state in which the cuff is enclosed; and a lock member that locks a part of the case members to maintain the open state and the stowed state.

### BRIEF DESCRIPTION OF THE DRAWINGS

FIG. 1 is a schematic of an overall configuration of a blood pressure monitor according to embodiment 1 of the present invention;

FIG. 2 is an exploded perspective view of the blood pressure monitor according to embodiment 1 of the present invention;

FIG. 3 is a cross-sectional view of a first locking mechanism according to embodiment 1 of the present invention;

FIG. 4 is a cross-sectional view of a second locking mechanism according to embodiment 1 of the present invention;

FIG. 5A is an exploded perspective view of a torque hinge according to embodiment 1 of the present invention;

FIG. 5B is an explanatory diagram explaining insertion of the torque hinge according to embodiment 1 of the present invention;

FIG. 6 is an exploded perspective view of a cuff according to embodiment 1 of the present invention;

FIG. 7 is an explanatory diagram of a display of the blood pressure monitor according to embodiment 1 of the present invention;

FIG. 8 is a functional block diagram of the blood pressure monitor according to embodiment 1 of the present invention;

FIG. 9 is a schematic explaining a manipulation for unlocking the first locking mechanism according to embodiment 1 of the present invention;

FIG. 10 is an oblique perspective view of the blood pressure monitor in a closed state according to embodiment 1 of the present invention;

FIG. 11 is a schematic illustrating a locking mechanism of a second locking mechanism according to embodiment 1 of the present invention;

FIG. 12 is a schematic of the second locking mechanism in a locked state according to embodiment 1 of the present invention;

FIG. 13 is a schematic illustrating a mechanism for unlocking the second locking mechanism according to embodiment 1 of the present invention;

FIG. 14 is a schematic of an overall blood pressure monitor according to embodiment 2 of the present invention; and

FIG. 15 is a cross-sectional view of the blood pressure monitor in a closed state according to embodiment 2 of the present invention.

### DETAILED DESCRIPTION OF THE PREFERRED EMBODIMENTS

Referring to the accompanying drawings, exemplary embodiments according to the present invention are explained in detail below.

Referring to the accompanying drawings, exemplary embodiments according to the present invention are explained in detail below.

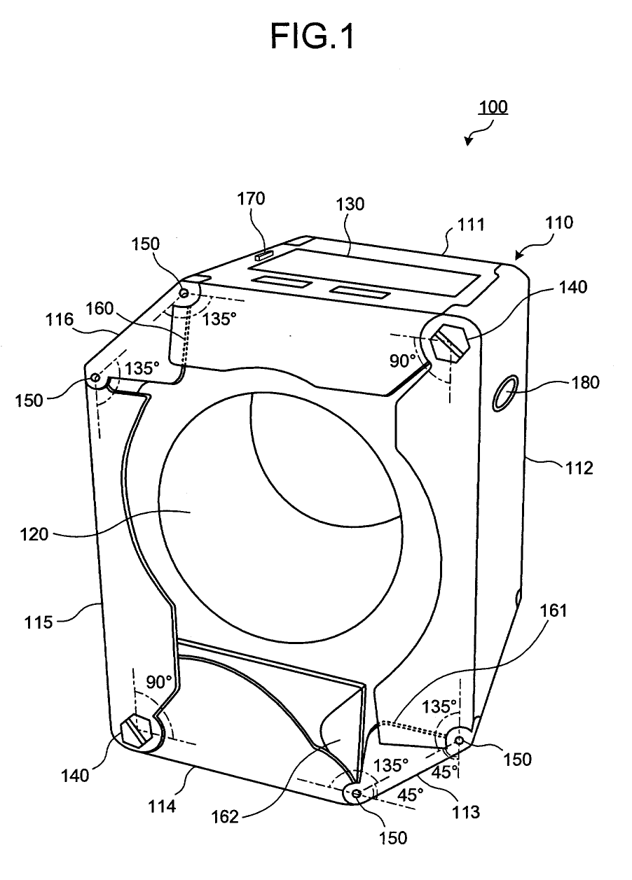

FIG. 1 is a schematic of an overall configuration of a blood pressure monitor according to embodiment 1 of the present invention. A blood pressure monitor 100 according to embodiment 1 of the present invention is collapsible and transformed between a collapsed, stowed state and an open state when in use. The open state is shown in FIG. 1. The stowed state will be explained hereinafter.

The blood pressure monitor 100 includes a housing 110, a cuff 120, and a display unit 130. The housing 110 includes a first case member 111 that is a cover, a second case member 112 that is a side plate, a third case member 113 that is an intermediate member, a fourth case member 114 that is a main body, a fifth case member 115 that is a side plate, and a sixth case member 116 that is an intermediate member. The intermediate member constitutes a height component of a rectangular shape in the stowed state that will be explained hereinafter.

The case members 111 to 116 are connected with one another by plural torque hinges 140 or plural rotation hinges 150. The first, the second, and the fourth case members 111, 112 and 114 respectively include slide recesses 160, 161, and 162 at positions where the case members contact and overlap with each other to prevent interference with the adjacent case member.

The cuff 120 is disposed in the case members 111 to 116 that are connected. The display unit 130 is disposed on the first case member 111, and positioned on an upper aspect of the housing 110 in the open state. Although explained in detail hereinafter, a reference character 170 represents a first button that is a part of a first locking mechanism for maintaining the open state, and a reference character 180 represents a second button that is a part of a second locking mechanism for maintaining the stowed state.

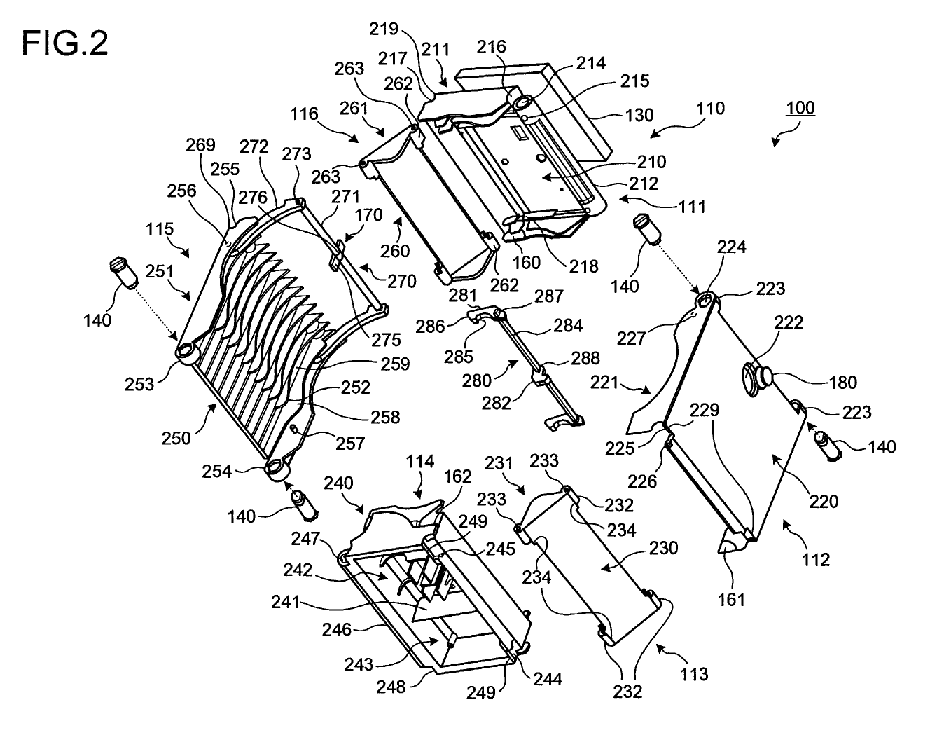

With reference to FIG. 2, a detailed configuration of the blood pressure monitor 100 is explained. FIG. 2 is an exploded perspective view of the blood pressure monitor 100 according to embodiment 1 of the present invention. The blood pressure monitor 100 according to embodiment 1 includes a first locking mechanism 270 and a second locking mechanism 280 in the case members 111 to 116 that constitute the housing 110.

The first case member 111 is a cover, disposed on the uppermost aspect, and opposing the fourth case member 114 that is the main body. The second and the fourth case members 112 and 114 that are side plates are disposed between the first and the fourth case members 111 and 114. The third case member 113 is an intermediate member provided between the second and the fourth case members 112 and 114. The sixth case member 116 is an intermediate member provided between the first and the fifth case members 111 and 115.

The first case member 111 includes a top plate 210 and a sidewall 211. A housing that houses the display unit 130 is provided on a surface of the top plate 210, and the display unit 130 is mounted therein. A cylinder 212 with openings at both ends thereof is provided at one end of the top plate 210. An insertion hole 214 for inserting the torque hinge 140 is formed at the opening at each end of the cylinder 212.

A fixing hole 215, into which a fixing member for preventing rotation of the torque hinge 140 is inserted, is formed near each end of the cylinder 212 on the outer surface. A longitudinal length of the cylinder 212 is shorter than that of the top plate 210, a connector 216 connected with the second case member 112 is formed on each end of the cylinder 212. A notch 217 is formed at the other end of the top plate 210. An insertion hole (not shown), into which the rotation hinge 150 is inserted, is formed in the notch 217. A stopper receiving unit 219 for receiving a stopper of the sixth case member 116 in the open state, explained hereinafter, is formed on the top plate 210 side of the notch 217.

The sidewall 211 includes a supporter 218 that supports the first locking mechanism 270. The sidewall 211 includes the slide recess 160 that contacts a sidewall 261 of the sixth case member 116 upon a closing or an opening of the blood pressure monitor 100 explained hereinafter.

The second case member 112 includes a planar section 220 and a sidewall 221. The planar section 220 includes a housing hole 222 for housing a second button 180 that is a part of the second locking mechanism 280. A non-depicted support rib is provided on the inside of the planar section 220. The support rib will be explained in detail with the fifth case member 115 explained hereinafter. A protrusion 223 protruding from the planar section 220 and the sidewall 221 is provided at one end of the planar section 220. An insertion hole 224 for inserting the torque hinge 140 is provided at the protrusion 223. The insertion hole 224 has a similar shape as one end of the torque hinge 140, for example, a hexagonal shape that can be fit to one end of the torque hinge 140.

A notch 225 is formed at other end of the planar section 220. An insertion hole 226 for inserting the rotation hinge 150 is formed at the notch 225. A stopper receiving unit 229 that receives a stopper 234 of the third case member 113 explained hereinafter is formed on the planar section 220 side of the notch 225.

A supporter 227 that supports the second locking mechanism 280 is formed on the sidewall 221. The slide recess 161 that contacts a sidewall 231 of the third case member 113 upon the closing or the opening of the blood pressure monitor 100 is formed on the sidewall 221.

The third case member 113 includes a plain 230 and a substantially triangular sidewall 231. A protrusion 232 protruding from the plain 230 and the sidewall 231 is formed on the base of the sidewall 231. An insertion hole 233, into which the rotation hinge 150 is inserted, is formed at the protrusion 232. A stopper 234 is formed at each of the four corners of the plain 230 and protruding from the plain 230.

The fourth case member 114 includes a non-depicted bottom plate and a sidewall 240. A partition 241 is provided in the fourth case member 114 to partition the fourth case member 114 into a pump compartment 242 and a battery compartment 243. A notch 244 is formed at one end of the bottom plate by cutting a part of the bottom plate and the sidewall 240. An insertion hole 245 for inserting the rotation hinge 150 is formed at the notch 244. A stopper receiving unit 249 that receives a stopper 234 of the third case member 113 in the open state is formed on the bottom plate side of the notch 244.

A cylinder 246 with openings at both ends thereof is provided on the other end of the bottom plate. An insertion hole 247 for inserting the torque hinge 140 is formed at each end of the cylinder 246. A fixing hole (not shown) for inserting a fixing member for preventing the rotation of the torque hinge 140 is formed on the outer surface in near each end of the cylinder 246.

A longitudinal length of the cylinder 246 is shorter than that of the bottom plate, and a connector 248 that connects with the fifth case member 115 is formed on each end of the cylinder 246. A slide recess 162 that contacts the sidewall 231 of the third case member 113 in the closing or the opening of the blood pressure monitor 100 is formed on the sidewall 240.

The fifth case member 115 includes a planar section 250 and a sidewall 251. A support rib 252 is provided on the planar section 250. The rib 252 is formed in a circular arc shape that fits the outer circumference of the cuff 120 in the open state. Although not shown, the rib of the second case member 112 has a similar configuration. A protrusion 253 protruding from the sidewall 251 is provided on one end of the planar section 250. An insertion hole 254 for inserting the torque hinge 140 is provided at the protrusion 253. Similar to the insertion hole 224 of the second case member 112, the insertion hole 254 is formed in a hexagonal shape.

A notch 255 is formed on the other end of the planar section 250. An insertion hole (not shown) for inserting the rotation hinge 150 is formed at the notch 255. A stopper receiving unit 269 that receives, in the open state, a stopper (not shown) of the sixth case member 116 explained hereinafter is formed on the planar section 250 side of the notch 255.

A first locking protrusion 256 by which the first locking mechanism 270 locks and a second locking protrusion 257 by which the second locking mechanism 280 locks are provided on the sidewall 251 of the fifth case member 115. A protruding wall 258 is provided between the outermost rib 252 and the sidewall 251. A cuff fit recess 259 that fits the cuff 120 is provided between the protruding wall 258 and the outermost rib 252. A similar cuff fit recess is formed on the inside of the second case member 112.

The sixth case member 116 has a configuration similar to that of the third case member 113, and includes a planar section 260 and a substantially triangular sidewall 261. A protrusion 262 protruding from the planar section 260 and the sidewall 261 is provided on the base of the sidewall 261. An insertion hole 263 for inserting the rotation hinge 150 is formed at the protrusion 262. A stopper (not shown) is formed at the four corners of the planar section 260 by a cutting of the planar section 260.

Connection of the case members 111 to 116 is explained. The first and the second case members 111 and 112 are connected with the torque hinge 140. The first and the second case members are connected by fitting the connector 216 of the first case member 111 to the protrusion 223 of the second case member 112, and inserting the torque hinge 140 into the insertion hole 214 of the first case member 111 and the insertion hole 224 of the second case member 112.

The torque hinge 140, as a pivot, rotatably connects the first case member 111 and the second case members 112, and applies a predetermined force to the first and the second case members 111 and 112. Therefore, the first and the second case members 111 and 112 are rotatable about the torque hinge 140 as the pivot, and the predetermined force is applied to the both case members 111 and 112 to form a predetermined angle (90°) between the case members 111 and 112 about the pivot.

The second and the third case members 112 and 113 are connected with the rotation hinge 150. The second and the third case members 112 and 113 are connected by fitting the notch 225 of the second case member 112 to the protrusion 232 of the third case member 113, and inserting the rotation hinge 150 into the insertion hole 226 of the second case member 113 and the insertion hole 233 of the third case member 113.

The rotation hinge 150 does not apply a force to the second and the third case members 112 and 113, and enables the second and the third case members 112 and 113 to rotate thereabout as a pivot in response to an externally applied force. Thus, the second and the third case members 112 and 113 are rotatable about the rotation hinge 150.

The stopper 234 of the third case member 113 fits the stopper receiving unit 229 of the second case member 112 so that the angle formed by the second and the third case members 112 and 113 rotating about the rotation hinge 150 does not exceed a predetermined angle in the open state. Specifically, in the open state shown in FIG. 1, the angle formed by the second and the third case members 112 and 113 rotating about the rotation hinge 150 does not exceed a predetermined angle (135°).

Although explained in detail with respect to the stowed state hereinafter, when the second and the third case members 112 and 113 rotate about the rotation hinge 150 to the predetermined angle, the slide recess 161 formed on the sidewall 221 of the second case member 112 and the sidewall 231 of the third case member 113 lock, thereby restricting the rotation, and preventing the further rotation to an angle less than a predetermined angle.

The connections of the third to the sixth case members 113 to 116 are similar to the connections of the first to the third case members 111 to 113 explained above. Specifically, the fourth and the fifth case members 114 and 115 are connected with the torque hinge 140. The third and the fourth case members 113 and 114, the fifth and the sixth case members 115 and 116, and the sixth and the first case members 116 and 111 are respectively connected with the rotation hinges 150.

In the open state shown in FIG. 1, due to the interposition of the intermediate members (the third and sixth members 113 and 116), the angle formed between the intermediate member and the case member connected with the intermediate member is 135°, and is supplementally explained. Although a case of the third case member as the intermediate member is explained, the same is applicable to the sixth case member 116.

The sidewall 231 of the third case member 113 is formed in a substantially isosceles right triangular shape. The third case member 113 rotates about two pivot positions through the rotation hinges 150, and connects with the second and fourth case members 112 and 114. In the open state shown in FIG. 1, the second and the fourth case members are disposed perpendicular to each other, and rotatably connected with the third case member by the rotation hinge 150. As a result, the third case member 113 is disposed obliquely at an identical angle with respect to the second and the fourth case members.

Therefore, as shown in FIG. 1, the third case member 113 is angled at 45° with respect to the horizontal plane. As a result, the third case member 113 is disposed such that the angle formed by the second and the third case members 112 and 113 and by the third and the fourth case members 113 and 114 is 135° that is a sum of the acute angles (45°) of the isosceles right triangle.

Thus, since the intermediate members are disposed, a stable shape can be maintained in the open state. The length of the bottom plate of the intermediate member can be shortened, and a height component can be shortened in the stowed state explained hereinafter.

The first locking mechanism is explained. The first locking mechanism 270 locks the first and the fifth case members 111 and 115 to maintain the open state. The first locking mechanism 270 includes a rotation bar 271, a pair of first arm members 272 (first locking members), and a first button 170.

The rotation bar 271 is supported by the supporter 218 of the first case member 111. The first arm members 272 are disposed at both ends of the rotation bar 271. The first arm member 272 is formed in a substantially circular arc shape, and includes a hook (not shown) at one end and an insertion hole 273 for inserting the rotation bar 271 at other end. The insertion hole 273 for inserting the rotation bar 271 is fixed to the rotation bar 271, interlocking the rotation of the rotation bar 271 with the first arm member 272.

The first button 170 is disposed at substantially the center of the rotation bar 271. The first button 170 includes an insertion hole 275 for inserting the rotation bar 271 is formed at the center of a plate member 275. One end of the first button 170 protrudes from the first case member 111 on the side of the display unit 130 as shown in FIG. 1. The insertion hole 276 into which the rotation bar 271 is inserted is fixed to the rotation bar 271, interlocking the rotation of the rotation bar 271 with the first button 170.

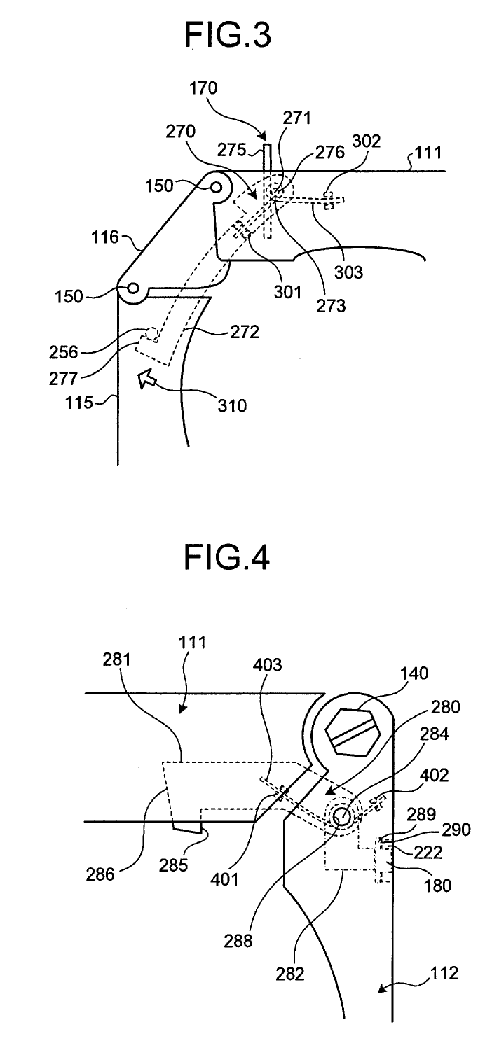

With reference to FIG. 3, the first locking mechanism 270 is explained in detail. FIG. 3 is a cross-sectional view of the first locking mechanism according to embodiment 1 of the present invention. The first locking mechanism 270 in the open state is shown in FIG. 3.

The first locking mechanism 270 includes a hinge spring 303, one end of which is fixed by an arm-side fixing member 301, and other end of which is fixed by a case-side fixing member 302, and a center roll of which is loosely wound about the rotation axis 271. The hinge spring applies a force to the first arm member 272 in the direction indicated by an arrow 310, the hook 277 locks at the first locking protrusion 256 provided on the fifth case member 115 in the open state of the blood pressure monitor 100.

With reference to FIG. 2, the second locking mechanism 280 is explained. The second locking mechanism 280 shown in FIG. 2 locks the second and the fifth case members 112 and 115 to maintain the stowed state.

The second locking mechanism 280 includes a second arm member 281 (second locking member), a rotation transferring member 282, a second button 180, and a rotation bar 284. The second arm member 281 is disposed on each end of the rotation bar 284. The second arm member 281 includes a hook 285 at one end, and an oblique portion 286 on the back side of the hook 285. The second arm member 281 includes, at other end, an insertion hole 287 for inserting the rotation bar 284. The insertion hole 287 for inserting the rotation bar 284 is fixed to the rotation bar 284, interlocking the rotation of the rotation bar 284 with the second arm member 281.

The rotation transferring member 282 is formed in an L-shape, and includes an insertion hole 288 at one end. The rotation transferring member 282 is disposed at substantially the center of the rotation bar 284, one end thereof is fixed to the rotation bar 284, and rotates together with the rotation bar 284. The other end of the rotation transferring member 282 is connected with the second button 180. The rotation bar 284 connects the second arm member 281 with the rotation transferring member 282, and is supported by the supporter 227 of the second case member 112.

With reference to FIG. 4, the second locking mechanism 280 is explained. FIG. 4 is a cross-sectional view of the second locking mechanism according to embodiment 1 of the present invention. The second locking mechanism 280 in the open state is shown in FIG. 4. The second locking mechanism 280 includes a hinge spring 403, one end of which is fixed by an arm-side fixing member 401, other end of which is fixed by a case-side fixing member 402, and a center roll of which is loosely wound about the rotation bar 284.

The rotation transferring member 282 is connected with the second button 180. A guard member 289 contacts the bottom plate 290 of the housing hole 222 provided on the second case member 112, preventing the second button 180 from slipping off and maintaining the second button 180 disposed at a height flush with the surface of the second case member 112.

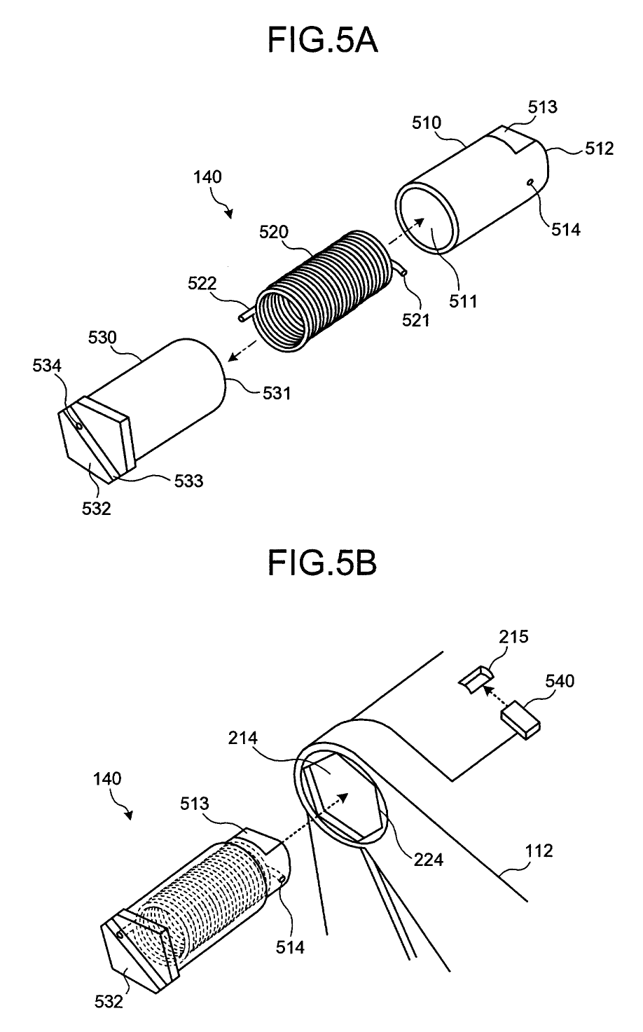

With reference to FIG. 5A, a configuration of the torque hinge 140 is explained. FIG. 5A is an exploded perspective view of the torque hinge according to embodiment 1 of the present invention. The torque hinge 140 includes a first cylindrical member 510, an elastic spring 520, and a second cylindrical member 530.

The first cylindrical member 510 is formed in a cylindrical shape, and includes an opening 511 at one end, and a sealed portion 512 at other end. The sealed portion 512 end of the first cylindrical member 510 is cut to have a D-shaped cross section, a planar section 513, the flat portion of the cross section, is formed on a part of the outer circumference. A lock hole 514 for locking the elastic spring 520 is formed on the outer circumference at the sealed portion 512 side of the first cylindrical member 510.

The elastic spring 520 is formed in a spiral shape. Both ends of the elastic spring 520 protrude therefrom, one end becomes a first locking portion 521 that locks at the first cylindrical member 510, and the other end becomes a second locking portion 522 that locks at the second cylindrical member 530.

The second cylindrical member 530 is formed in a cylindrical shape, and includes an opening 531 at one end, and a head 532 at the other end that is formed in a hexagonal shape. A groove 533 is formed on the head 532. A lock hole 534 for locking the second locking portion 522 of the elastic spring 520 is formed on the groove 533.

The first cylindrical member 510, the elastic spring 520, and the second cylindrical member 530 are combined to form the torque hinge 140 according to embodiment 1. Specifically, the elastic spring 520 is inserted into the opening 511 of the first cylindrical member 510, and the first locking portion 521 of the elastic spring 520 is inserted into the lock hole 514.

The first cylindrical member 510 is inserted into the second cylindrical member 530 with the elastic spring 520 inserted. At this time, the second locking portion 522 of the elastic spring 520 is inserted into the lock hole 534 of the second cylindrical member 530 to be locked. Grease is applied to the inner circumference of the first and the second cylindrical members 510 and 530, and a surface of the elastic spring 520.

Therefore, the first and the second cylindrical members 510 and 530 are relatively rotatable, and the elastic spring 520 causes a force against the rotation direction.

FIG. 5B is an explanatory diagram explaining insertion of the torque hinge according to embodiment 1 of the present invention. Although a case in which the first and the second case members 111 and 112 are connected with the torque hinge 140 is explained, the same is applicable to a case in which the fourth and the fifth case members 114 and 115 are connected. The torque hinge 140 is inserted in the open state. The open state indicates a state in which the angle formed by the first and the second case members 111 and 112 that rotate about the torque hinge 140 as a pivot is 90°, for example.

The torque hinge 140 is inserted into the insertion hole 214 of the first case member 111 and the insertion hole 224 of the second case member 112. When the torque hinge 140 is inserted, the D-shape cut portion of the torque hinge 140, specifically, the planar section 513 of the torque hinge 140 is disposed at a position corresponding to the fixing hole 215 of the first case member 111.

A fixing member 540 is inserted into the fixing hole 215, thereby fixing one end of the torque hinge 140. Meanwhile, the head 532 of the torque hinge 140 is fitted into the insertion hole 224 of the second case member 112 that is formed in a hexagonal shape, and fixed. As a result, the first and the second case members 111 and 112 are rotatable about the torque hinge 140 as a pivot.

Regarding the applied force, when the torque hinge 140 is inserted in the open state, for example, the force applied by the torque hinge 140 can be weakened in the open state. On the other hand, in a state transformed from the open state, for example, when the angle formed by the first and the second case members 111 and 112 that rotate about the torque hinge 140 as the pivot becomes greater than 90°, a force causing the angle to become 90°, which is the open state, is applied.

The torque hinge 140 according to embodiment 1 can adjust the force. Specifically, the force applied by the torque hinge 140 can be adjusted by rotating the head 532 left or right. More specifically, since the one end of the torque hinge 140 is fixed at the fixing member 540, the force applied by the torque hinge 140 can be adjusted according to the position (phase) of the head 532 on the other end. Therefore, the applied force can be weakened or strengthened by rotating the head 532 formed in a hexagonal shape in 60° intervals left or right.

The adjustment of the force applied by the torque hinge 140 is not limited to the method of rotating the torque hinge left or right. The force applied by the torque hinge 140 is relatively determined by the angle formed by the first and the second case members 111 and 112 rotating about the torque hinge 140 as a pivot. Therefore, if the torque hinge 140 is inserted with the angle formed by the first and the second case members 111 and 112 deviated from 90°, the force is adjusted by the margin deviating from 90°.

Additionally, if the head 532 is formed in an octagon shape, or a polygonal shape, the rotation range of the head 532 becomes small, further enabling fine adjustment of the applied force. On the other hand, to increase a holding force of the head 532, and to transfer the force applied by the torque hinge 140 to the insertion hole 224 steadily, the head 532 may be smaller than hexagonal, for example, quadrangular. Although the torque hinges 140 are used at two points on one side surface, at a total of four points on both surfaces in embodiment 1, the torque hinges 140 may be used for all the portions at which the rotation hinges 150 are disposed.

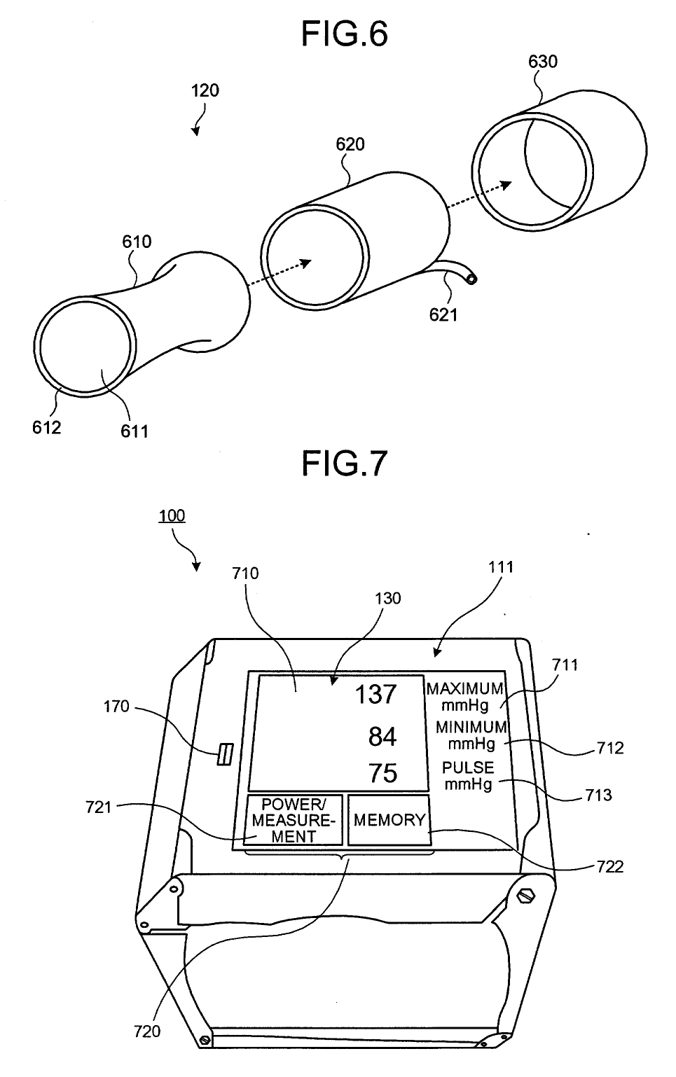

With reference to FIG. 6, a configuration of the cuff 120 is explained. FIG. 6 is an exploded perspective view of the cuff according to embodiment 1 of the present invention. The cuff 120 according to embodiment 1 includes a cuff ring 610, a blood-flow restricting bag 620, and a cuff band 630.

The cuff ring 610 is made of polyethylene, and includes two openings 611. An elastic ring 612 such as a resin ring and a coil-spring-like metal ring is provided, as a supporting member, on each end of the cuff ring 610.

The blood-flow restricting bag 620 is made of polyurethane, and connected with a non-depicted air pump through an inflation-deflation tube 621. The cuff band 630 is made of nylon, and prevents expansion of the inflated blood-flow restricting bag 620. The cuff band 630 has a circumferential extension rate much smaller than an axial extension rate.

The elastic ring 612 of the cuff 120 according to embodiment 1 fits the cuff fit groove 259 of the fifth case member shown in FIG. 2. Similarly, the elastic ring 612 fits the cuff fit groove of the second case member 112.

When the blood-flow restricting bag 620 of the cuff 120 according to embodiment 1 is inflated by the non-depicted air pump upon measurement, the cuff band 630 prevents the blood-flow restricting bag 620 from expanding outwards. Especially, the cuff band 630 has a circumferential expansion rate much smaller than the axial expansion rate, thereby restricting the outward expansion of the blood-flow restricting bag 620.

With reference to FIG. 7, the display unit 130 of the blood pressure monitor 100 according to embodiment 1 of the present invention is explained. FIG. 7 is an explanatory diagram of the display of the blood pressure monitor according to embodiment 1 of the present invention. The display unit 130 is housed on the first case member 111. The display unit 130 includes a screen 710 and an operation unit 720.

The screen 710 includes a maximum-blood-pressure display 711, a minimum-blood-pressure display 712, and a pulse display 713. The maximum-blood-pressure display 711 displays a maximum blood pressure. The minimum-blood-pressure display 711 displays a minimum blood pressure. The pulse display 713 displays a pulse.

The operation unit 720 includes a power/measurement button 721 and a memory button 722. The power/measurement button 721 turns the power on and off according to user manipulation thereof. The memory button 722 stores measured values, after measurement, into a memory according to user manipulation thereof. Power to the display unit 130 is supplied by the battery housed in the battery compartment 243 of the fourth case member 114 shown in FIG. 2.

The view of the display unit 130 shown in FIG. 7 is from that of a user when the user inserts his/her upper arm onto the blood pressure monitor 100 to measure blood pressure. Since the display unit 130 is mounted on the top plate 210 of the first case member 111, good visibility of the screen 710 can be achieved.

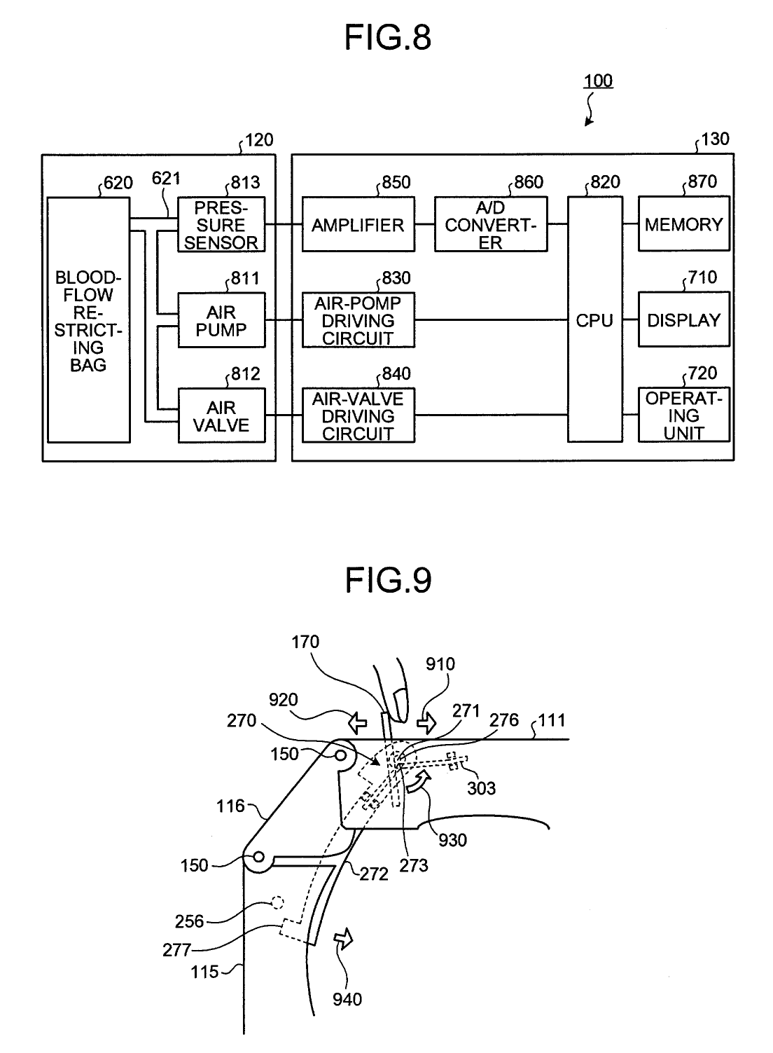

With reference to FIG. 8, a functional configuration of the blood pressure monitor 100 according to embodiment 1 of the present invention is explained. FIG. 8 is a functional block diagram of the blood pressure monitor according to embodiment 1 of the present invention. When a user inserts his/her arm onto the cuff, and depresses the power/measurement button 721, the blood pressure monitor 100 automatically inflates the cuff 120 to initiate the measurement.

The blood pressure monitor 100 includes the cuff 120 and a display unit 130. The cuff 120 includes the blood-flow restricting bag 620, an air pump 811, an air valve 812, and a pressure sensor 813. The blood-flow restricting bag 620 is connected to the air pump 811, the air valve 812, and the pressure sensor 813 through the inflation-deflation tube 621.

The air pump inflates the blood-flow restricting bag 620. Specifically, during measurement, the air pump 811 pumps compressed air into the blood-flow restricting bag 620 so that the pressure therein becomes a predetermined pressure. The air valve 812 maintains and reduces the pressure in the blood-flow restricting bag 620. Specifically, the air valve 812 maintains and deflates the blood-flow restricting bag 620 of high pressure compressed by the air pimp 811 during the measurement, and deflates the blood-flow restricting bag 620 to atmospheric pressure after the measurement. The pressure sensor 813 detects pressure in the blood-flow restricting bag 620 that varies during the measurement.

The display unit 130 includes a CPU 820, an air-pump driving circuit 830, an air-valve driving circuit 840, an amplifier 850, an A/D converter 860, a memory 870, the screen 710, and the operation unit 720. The CPU 820 controls the blood pressure monitor 100 overall. The air-pump driving circuit 830 controls driving of the air pump 811 under the control of the CPU 820. The air-valve driving circuit 840 controls open and closed states of the air valve 812 under the control of the CPU 820. The amplifier 850 amplifies a pressure detection signal of the blood-flow restricting bag 620 output from the pressure sensor 813, and outputs the amplified signal to the A/D converter 860. The A/D converter 860 digitalizes the analog signal output from the amplifier 850, and outputs the digitalized signal to the CPU 820.

The CPU 820 controls the air pump 811, the air valve 812, and the pressure sensor 813 based on an instruction received by the operation unit 720 of the display unit 130, and outputs a measurement result to the screen 710. When the user manipulates the memory button 722, based on the measurement result displayed on the screen 710, the CPU 820 outputs the measurement result to the memory 870. Then, the memory 870 stores the measurement result.

A manipulation for unlocking the first locking mechanism according to embodiment 1 of the present invention is explained. FIG. 9 is a schematic explaining a manipulation for unlocking the first locking mechanism according to embodiment 1 of the present invention. The first locking mechanism 270 locks the first and the fifth case members 111 and 115 to maintain the open state. Specifically, as explained above, in the open state, the first arm member 272 of the first locking mechanism 270 locks at the first locking protrusion 256 by the force applied by the hinge spring 303. At this time, the hinge spring 303 applies a force to the first button 170 of the first locking mechanism 270 in the direction indicated by an arrow 910.

It is assumed that the user finished using the blood pressure monitor 100 now. When the user applies a force to the first button 170 in the direction indicated by an arrow 920, the first button 170 is inclined, in the direction indicated by the arrow 920, about the rotation bar 271 as the pivot. At this time, the insertion hole 276 of the first button 170 rotates in the direction indicated by an arrow 930. The rotation bar 271 rotates in the direction indicated by the arrow 930 together with the insertion hole 276. At the same time, the insertion hole 273 of the first arm member 272 rotates in the direction indicated by the arrow 930 together with the rotation bar 271. Then, the first arm member 272 rotates about the rotation bar 271 as a pivot toward an arrow 940.

Thus, the lock hook 277 of the first arm member 272 is released from the first locking protrusion 256, thereby unlocking the lock of the first locking mechanism 270. When the first locking mechanism 270 is unlocked, the blood pressure monitor 100 is in a collapsible state. The blood pressure monitor 100 is collapsed by the user depressing the first case member 111 downwards.

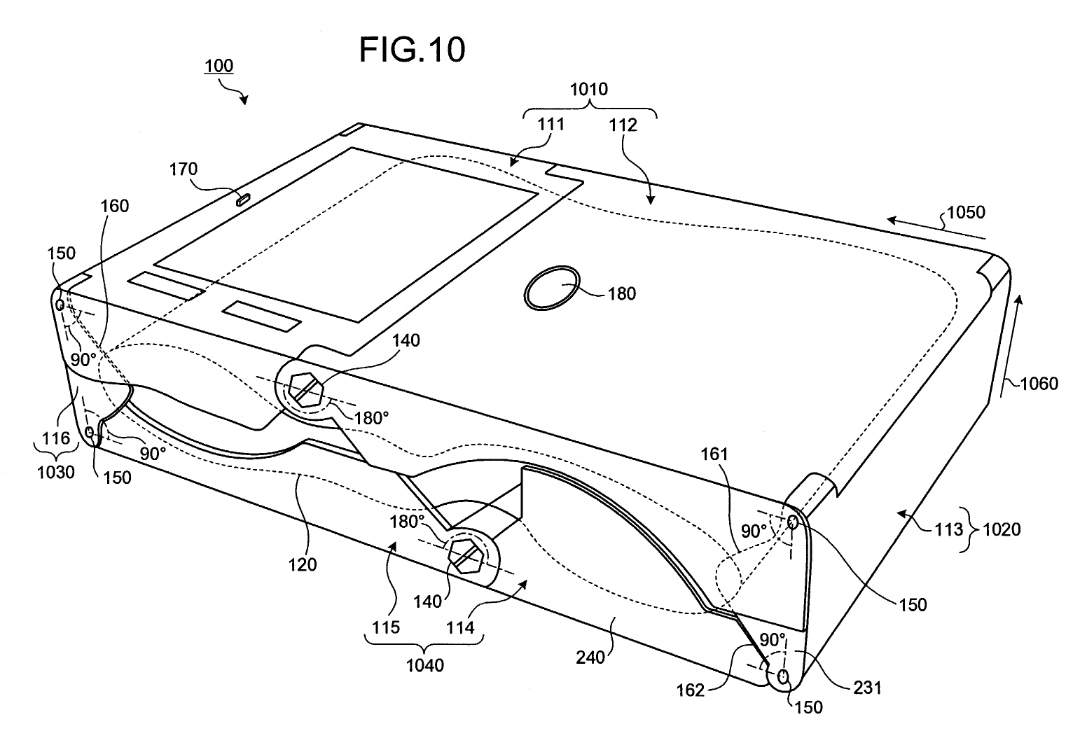

FIG. 10 is an oblique view of the blood pressure monitor in the stowed state according to embodiment 1 of the present invention. In the stowed state, the first and the second case members 111 and 112 form a top plate 1010, the third case member 113 forms a side plate 1020, the sixth case member 116 forms a side plate 1030, and the fourth and the fifth case members 114 and 115 form a bottom plate 1040.

In other words, in the direction indicated by an arrow 1050, the dimension of the first and the second case members 111 and 112 is identical to the length of the fourth and fifth case members 114 and 115. In the direction indicated by an arrow 1060, the dimension of the third case member 113 is identical to that of the sixth case member 116.

The transformation of the blood pressure monitor 100 from the open state to the stowed state is explained. Upon the closing transformation, each of the case members 111 to 116 rotates about the torque hinges 140 and the rotation hinges 150 as the pivots to be collapsed. Specifically, when a user depresses the first case member 111 against the force applied by the torque hinge 140, the angle formed by the first and the second case members 111 and 112 rotating about the torque hinge 140 as a pivot changes to 180°. Similarly, the angle formed by the fourth and the fifth case members 114 and 115 rotating about the torque hinge 140 as a pivot changes to 180°.

On the other hand, each of the case members 111 to 116 that are connected with the rotation hinge 150 rotates about the rotation hinge 150. The movements of the case members 111 to 116 about the rotation hinge 150 as a pivot is explained with an example of the movements of the third and the fourth case members 113 and 114. When the angle formed by the third and the fourth case members 113 and 114 rotating about the rotation hinge 150 changes from the open state shown in FIG. 1 (135°) to a predetermined angle (90°), the slide recess 162 formed on the sidewall 240 of the fourth case member 114 and the sidewall 231 of the third case member 113 lock, thereby restricting the rotation.

Similarly, with respect to the slide recess 160 of the first case member 111 and the sidewall 261 of the sixth case member 116, the angle formed by the slide recess 160 of the first case member 111 and the sixth case member 116 stops at 90°, thereby restricting the rotation.

Thus, since the angle formed by each of the case members 111 to 116 that are connected by the rotation hinge 150 and rotating about each rotation hinge 150 cannot be less that 90°, the blood pressure monitor 100 can be in a rectangular shape in the stowed state. Therefore, the blood pressure monitor 100 does not become in a parallelogram shape that is a distorted rectangular shape, and can maintain the rectangular shape having space to house the cuff 120, using the least dimension of the third and the sixth case members 113 and 116 that are the height components of the blood pressure monitor in the stowed state. Since the blood pressure monitor 100 is in the rectangular shape having no unnecessary protrusions, the blood pressure monitor 100 has not only good storability, but also a stable shape, thereby enhancing rigidity.

In the stowed state of the blood pressure monitor 100, the elastic ring 612 is deformed, and the cuff 120 is housed in the case members 111 to 116. In the stowed state of the blood pressure monitor 100, the second and the fifth case members 112 and 115 partially oppose each other. The stowed state is maintained by the second locking mechanism 280 locking the second and the fifth case members 112 and 115.

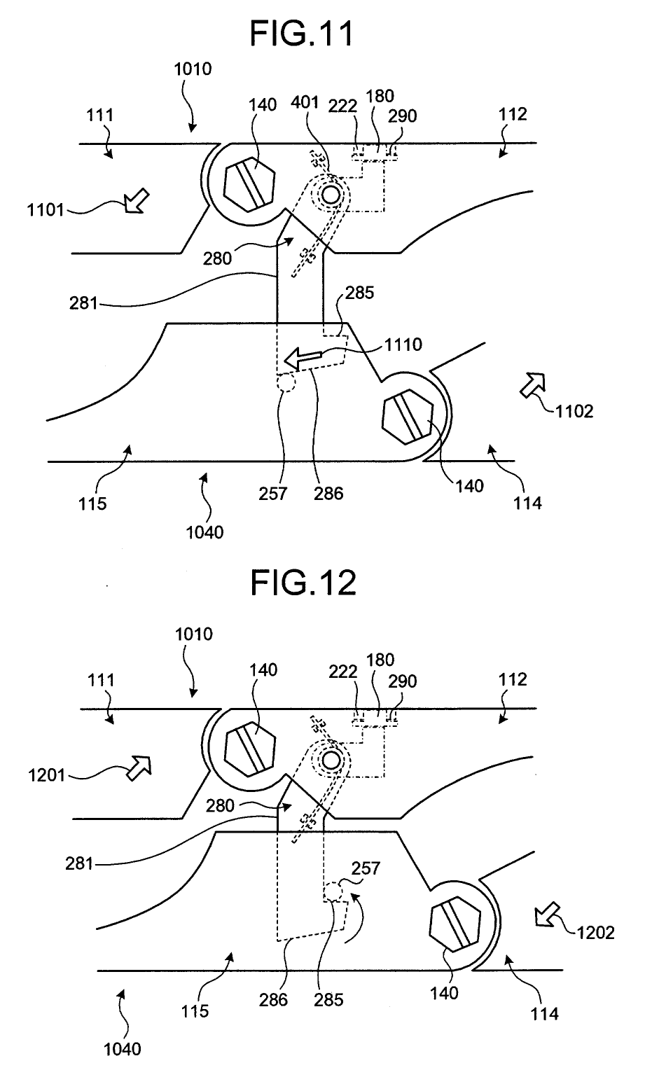

FIG. 11 is a schematic illustrating a locking mechanism of the second locking mechanism according to embodiment 1 of the present invention. Upon the closing transformation of the blood pressure monitor 100, the angle formed by the first and the second case members 111 and 112 rotating about the torque hinge 140 as a pivot increases, thereby forming the top plate 1010.

Similarly, the angle formed by the fourth and the fifth case members 114 and 115 rotating about the torque hinge 140 as a pivot increases, thereby forming the bottom plate 1040. At this time, the top plate 1010 moves in the direction indicated by an arrow 1101 with respect to the bottom plate 1040, and the bottom plate 1040 moves in the direction indicated by an arrow 1102 with respect to the top plate 1010.

When the blood pressure monitor 100 is collapsed to predetermined degree, the oblique portion 286 of the second arm member 281 contacts the second locking protrusion 257 provided on the fifth case member 115. When the blood pressure monitor 100 is further collapsed, the oblique portion 286 slides on the second locking protrusion 257 as indicated by an arrow 1110, and the second arm member 281 moves in the direction indicated by the arrow 1110. When the hook 285 of the second arm member 281 moves beyond the second locking protrusion 257, the hook locks therewith.

FIG. 12 is a schematic of the second locking mechanism in a locked state according to embodiment 1 of the present invention. The second locking mechanism 280 locks the second and the fifth case members 112 and 115 by the second arm member 281 locking at the second locking protrusion 257. At this time, the force applied by the torque hinge 140 is relatively applied to the top plate 1010 in the direction indicated by an arrow 1201, and relatively to the bottom plate 1040 in the direction indicated by an arrow 1202. Thus, according to embodiment 1, the second locking mechanism 280 locks the second and the fifth case members 112 and 115, thereby maintaining the stowed state of the blood pressure monitor 100 resisting the force applied by the torque hinge 140.

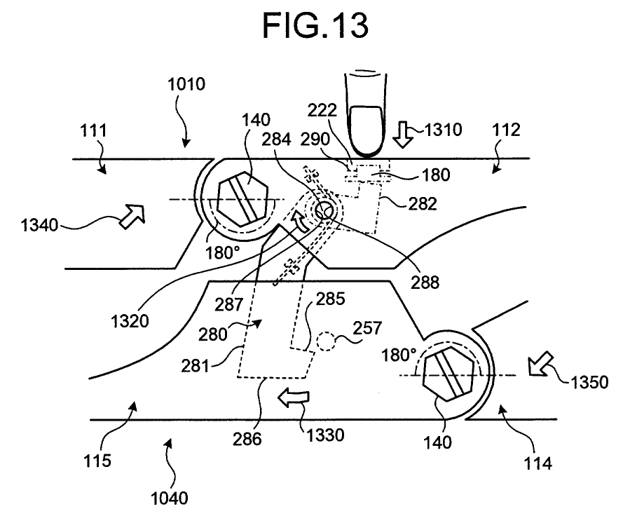

The opening transformation of the blood pressure monitor 100 from the stowed state to the open state is explained. FIG. 13 is a schematic illustrating a mechanism for unlocking the second locking mechanism according to embodiment 1 of the present invention. Assuming that a user depresses the second button 180, i.e., applies a force in the direction indicated by an arrow 1310 now, the rotation transferring member 282 inclines about the rotation bar 284 as a pivot, the insertion hole 288 of the rotation transferring member 282 rotates in the direction indicated by an arrow 1320. The rotation bar 284 rotates in the direction indicated by the arrow 1320 together with the insertion hole 288. The insertion hole 287 of the second arm member 281 rotates in the direction indicated by the arrow 1320 together with the rotation bar 284. The second arm member 281 rotates about the rotation bar 284 as a pivot in the direction indicated by an arrow 1330. Thus, the hook 285 of the second arm member 281 is released from the second locking protrusion 257, thereby unlocking the second locking mechanism 280.

When the second locking mechanism 280 is unlocked, the force applied by the torque hinge 140 causes the blood pressure monitor 100 to transform from the stowed state to the open state shown in FIG. 1. Specifically, the top plate 1010 moves in the direction indicated by an arrow 1340 with respect to the bottom plate 1040, and the bottom plate 1040 moves in the direction indicated by an arrow 1350 with respect to the top plate 1010. At this time, due to the force applied by the torque hinge 140, the angle formed by the first and the second case members 111 and 112 rotating about the torque hinge as a pivot decreases from 180° to 90° as shown in FIG. 1. Similarly, the angle formed by the fourth and the fifth case members 114 and 115 rotating about the torque hinge 140 as a pivot decreases from 180° to 90°.

Each of the case members connected with the rotation hinges 150 shown in FIG. 10 (specifically, the first and the sixth case members 111 and 116, the second and the third case members 112 and 113, the third and the fourth case members 113 and 114, and the fifth and the sixth members 115 and 116) rotates about the rotation hinge 150 such that each angle formed by the case members increases from 90° to 135°.

In the open state shown in FIG. 1, the force applied by the torque hinge 140 is weakened, and for example, the stopper 234 provided on the third case member 113 is received by the stopper receiving unit 229 of the second case member 112 and the stopper receiving unit 249 of the fourth case member 114, thereby restricting the rotation of the second and the third case members 112 and 113 and of the third and the fourth case members 113 and 114. Similarly, due to the stopper provided on the sixth case member 116, the rotation of the fifth and the sixth members 115 and 116, and the first and the sixth case members 111 and 116 are restricted.

In the open state, due to the first locking mechanism 270 of the blood pressure monitor 100, the first and the fifth case members 111 and 115 are locked. Since the elastic ring 612 fits the cuff fit groove of the second case member 112 and the cuff fit groove 259 of the fifth case member 115, the cuff 120 is formed in the cylindrical shape upon the opening transformation of the case members 111 to 116. Thus, the blood pressure monitor 100 is transformed from the stowed state to the open state.

Although a user inclines the board member 275 directly to rotate the rotation bar 271 with regard to the first button 170 of the first locking mechanism 270 according to embodiment 1, the configuration is not limited hereto, any configuration may be used as long as the rotation bar 271 can be rotated by a simple manipulation. Specifically, for example, as shown in FIG. 4, rotation bar 271 may be rotated by depressing the second button 180 used for the second locking mechanism 280 and the rotation transferring member 282.

Although the power is turned on and off by the power/measurement button 721 of the display unit 130 as shown in FIG. 7 in embodiment 1, the method is not limited hereto, but may be turned on and off when the first locking mechanism 270 for maintaining the open state is locked or unlocked, or when the second locking mechanism 280 for maintaining the stowed state is locked or unlocked.

Specifically, in a case in which the power is turned off upon unlocking the first locking mechanism 270, for example, the power is turned off when the first button 170 is inclined by the manipulation of the first button 170. In a case in which the power is turned off when the second locking mechanism 280 is locked, for example, the power is turned off when the second arm member 281 locks at the lock protrusion 257.

In a case in which the power is turned on when the first locking mechanism 270 is locked, the power is turned on when the first button 170 is at a predetermined position and has a predetermined posture by locking the first locking mechanism 270. In this case, if the blood pressure monitor 100 is left for a long time with the power on, unnecessary battery consumption occurs. Therefore, when a predetermined period of time elapses after the power is turned on, the power may be turned off.

As explained above, according to the blood pressure monitor 100 according to embodiment 1 of the present invention, plural case members 111 to 116 are rotatably connected with one another, and circularly surrounds the cuff 120, enabling the plural case members 111 to 116 to be transformed between the stowed state and the open state. Therefore, both ends of each of the case members 111 to 116 are supported by one another, achieving miniaturization of the device without loss of strength.

According to the blood pressure monitor 100 according to embodiment 1 of the present invention, plural case members 111 to 116 that are connected with the torque hinge 140 and the rotation hinge 150 are included, among the plural case members 111 to 116, the display unit 130 is provided on the first case member 111 that is the cover, thereby achieving miniaturization of the device particularly in the stowed state, and enhancing the visibility of the screen 710.

According to the blood pressure monitor 100 according to embodiment 1 of the present invention, the plural case members 111 to 116 that surround the outer circumference of the cuff 120 are provided, the plural case members 111 to 116 are rotatably connected with the torque hinge 140 and the rotation hinge 150, and the plural case members 111 to 116 can be transformed between the open state in which the cuff 120 is open, and the stowed state in which the cuff 120 is enclosed. Furthermore, the first and the second locking mechanisms 270 and 280 are included to maintain the open state in which the cuff 120 is open, and the stowed state in which the cuff 120 is enclosed.

Therefore, miniaturization of the device is achieved and a stable shape can be maintained during use, particularly miniaturization of the device can be achieved in the stowed state. Additionally, since the device becomes compact, flat and rectangular in the stowed state, storability is enhanced when plural blood pressure monitors 100 are accumulated, which is effective for storing and conveying the monitors in storage, etc.

According to the blood pressure monitor 100 according to embodiment 1 of the present invention, the first and the second case members 111 and 112, and the fourth and the fifth case members are connected by the torque hinges 140. Therefore, in the stowed state, a force is applied in a direction that transforms the device into the open state, enabling the simple transformation from the stowed state to the open state.

According to the blood pressure monitor 100 according to embodiment 1 of the present invention, the head 532 of the torque hinge 140, and the insertion hole 224 of the case member (for example, the second case member 112) are formed in a hexagonal shape to adjust the force applied by the torque hinge 140, enabling torque adjustment of the torque hinge 140. Therefore, the device can be transformed from the stowed state to the open state at an ideal speed for a user.

According to the blood pressure monitor 100 according to embodiment 1 of the present invention, the cuff 120 includes the blood-flow restricting bag 620 outside the cuff ring 610, and the cuff band 630 outside the blood-flow restricting bag 620. Therefore, since the cuff band 630 restricts the expansion of the blood-flow restricting bag 620 when the blood-flow restricting bag 620 is inflated by air, the pressure of the blood-flow restricting bag 620 can be effectively increased to compress the inserted arm.

According to the blood pressure monitor 100 according to embodiment 1 of the present invention, the cuff band 630 has a circumferential expansion rate lower than an axial expansion rate. Therefore, circumferential expansion of the blood-flow restricting bag 620 to which a circumferential force greater than an axial force is applied upon the measurement is prevented, thereby enabling effective increase in the pressure of the blood-flow restricting bag 620 to compress the inserted arm.

According to the blood pressure monitor 100 according to embodiment 1 of the present invention, the cuff ring 610 includes two openings on which the elastic rings 612 are provided, and the elastic ring 612 fits the cuff fit groove of the second case member 112, and the cuff fit groove 259 of the fifth case member. Therefore, the cuff 120 can be closed or opened according to the closing transformation and the opening transformation of the case members 111 to 116. Additionally, a user can be saved trouble, such as adjustment of the position and/or the shape of the cuff 120.

According to the blood pressure monitor 100 according to embodiment 1 of the present invention, the first locking mechanism is provided. Specifically, in the open state of the blood pressure monitor 100, the first arm member 272 disposed between the first and the fifth case members 111 and 115 that are connected via the sixth case member is provided on the first case member 111, and the first locking protrusion 256 at which the first arm member 272 locks is provided on the fifth case member 115. Therefore, the open state can be maintained, and collapsing of the blood pressure monitor 100 can be prevented during the measurement. Additionally, the blood pressure monitor 100 can be prevented from falling to the opposite side.

According to the blood pressure monitor 100 according to embodiment 1 of the present invention, the first locking mechanism 270 includes the first button 170 for unlocking the lock of the first arm member 272. Therefore, a user can unlock the first locking mechanism 270 by a simple button manipulation.

According to the blood pressure monitor 100 according to embodiment 1 of the present invention, the second locking mechanism 280 is provided. Specifically, in the stowed state of the blood pressure monitor 100, the second arm member 281 disposed between the second and the fifth case members 112 and 115 is provided on the second case member 112, and the second locking protrusion 257 at which the second arm member 281 locks is provided on the fifth case member 115. Therefore, the stowed state of the blood pressure monitor 100 can be maintained, and unintentional transformation to the open state can be prevented.

According to the blood pressure monitor 100 according to embodiment 1 of the present invention, the second locking mechanism 280 includes the second button 180 for unlocking the lock of the second arm member 281. Therefore, a user can transform the blood pressure monitor 100 from the stowed state to the open state by a simple button manipulation.

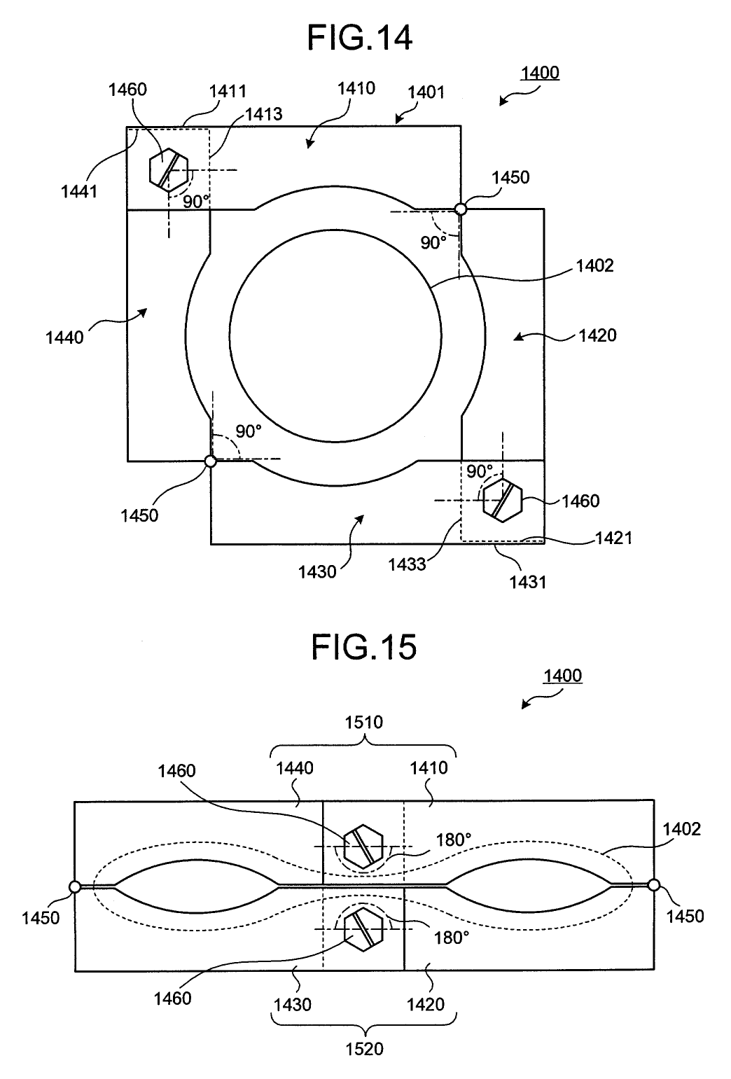

FIG. 14 is a schematic of an overall blood pressure monitor according to embodiment 2 of the present invention. A blood pressure monitor 1400 according to embodiment 2 of the present invention differs from that in embodiment 1 in that the four case members are used. Basic configurations of a cuff, a display, a first locking mechanism, a second locking mechanism, a torque hinge, and the like are identical to those in embodiment 1, omitting explanation thereof.

An open state of the blood pressure monitor 1400 is shown in FIG. 14. The blood pressure monitor 1400 includes a housing 1401, a cuff 1402, a non-depicted display. The housing 1401 includes a first case member 1410 that is to be a cover, and a second case member 1420 that is to be a side plate. Although not shown, a fit groove on which the cuff 1402 fits is formed inside the case members 1410 to 1440.

The first and the second case members 1410 and 1420 are connected with a rotation hinge 1450, and are rotatable about the rotation hinge 1450 as a pivot. Similarly, the third and the fourth case members 1430 and 1430 are connected with a rotation hinge 1450.

The second and the third case members 1420 and 1430 are connected by a torque hinge 1460, subjected to a force by the torque hinge 1460, and rotatable about the torque hinge 1460 as a pivot. Similarly, the fourth and the first case members 1440 and 1410 are connected by the torque hinge 1460.

Similar to embodiment 1, slide recesses 1413 and 1433 are respectively provided on an inside of the first and the third case members 1410 and 1430 so that these case members do not interfere with the adjacent case members 1420 and 1440. The angle formed by the first and the fourth case members 1410 and 1440 rotating about the torque hinge 1460 does not exceed a predetermined angle (90°). Specifically, a planar section 1411 of the first case member 1410 contacts a sidewall 1441 of the fourth case member 1440, thereby restricting the rotation. Similarly, a planar section 1431 of the third case member 1430 contacts a sidewall 1421 of the second case member 1420, thereby restricting the rotation.

The first case member 1410 includes the display on a surface thereof, and visibility is well maintained upon measurement. Similar to embodiment 1, the blood pressure monitor 1400 maintains the open state by the non-depicted locking mechanism locking the first and the fourth case members 1410 and 1440, for example.

With reference to FIG. 15, a closed state of the blood pressure monitor 1400 according to embodiment 2 is explained. FIG. 15 is a cross-sectional view of the blood pressure monitor in the stowed state according to embodiment 2 of the present invention. The blood pressure monitor 1400 becomes in the stowed state by a user unlocking the first locking mechanism and depressing the first case member 1410. In the stowed state, the first and the fourth case members 1410 and 1440 form a top plate 1510, and the second and the third case members 1420 and 1430 form a bottom plate 1520. In this state, similar to embodiment 1, the blood pressure monitor 1400 maintains the stowed state by the second locking mechanism locking the case members opposing each other, for example, the first and the second case members 1410 and 1420.

Transformation of the blood pressure monitor 1400 from the open state to the stowed state is explained. Upon the transformation from the open state to the stowed state, the blood pressure monitor 1400 is collapsed by each of the case members 1410 to 1440 rotating about the rotation hinge 1450 as a pivot. Specifically, after depressing a non-depicted first button of the first locking mechanism, a user pushes the first case member 1410 against the force applied by the torque hinge 1460. Then, the first and the fourth case members 1410 and 1440 rotate about the torque hinge 1460 as a pivot until the angle formed thereby changes from 90° as shown in FIG. 14 to 180°. Similarly, the second and the third case members 1420 and 1430 rotate about the torque hinge 1460 as a pivot until the angle formed by the second and the third case members 1420 and 1430 becomes 180°.

In contrast, the first and the second case members 1410 and 1420 that are connected by the rotation hinge 1450 rotate about the rotation hinge 1450 until the angle formed by the first and the second case members 1410 and 1420 changes from 90° to 0°. Similarly, the third and the fourth case members 1430 and 1440 that are connected by the rotation hinge 1450 rotate about the rotation hinge 1450 as a pivot until the angle formed by the third and the fourth case members 1430 and 1440 becomes 0°.

Although the torque hinge 1460 exerts a force opening the blood pressure monitor 1400 in the stowed state, the second locking mechanism locks the first and the second case members 1410 and 1420, thereby maintaining the stowed state of the blood pressure monitor 1400. In the stowed state of the blood pressure monitor 1400, elastic rings are transformed to house the cuff 1402 in the case members 1410 to 1440.

With regard to the opening transformation, similar to embodiment 1, when the second locking mechanism is unlocked, a force applied by the torque hinge 1460 causes the transformation from the stowed state to the open state. Upon the opening transformation of the blood pressure monitor 1400, since the elastic ring fits the fit groove, the cuff 1402 is transformed from the stowed state to a cylindrical shape in conjunction with the opening transformation of the case members 1410 to 1440.

Different from embodiment 1, the blood pressure monitor 1400 includes no case member forming a side plate. Specifically, the third and the sixth case members 113 and 116 as the intermediate members in embodiment 1 are not present. Therefore, case members having greater storage capacity than those in embodiment 1, i.e., case members having tall sidewalls are used for the case members 1410 to 1440.

As explained above, according to the blood pressure monitor 1400 according to embodiment 2 of the present invention, four case members 1410 to 1440 are rotatably connected and surround the cuff ring 1402 circularly, enabling the transformation between the stowed state in which the four case members 1410 to 1440 are collapsed and the open state in which the four case members are opened. Therefore, each of the case members is supported by one another, enabling miniaturization of the device without loss of strength.

According to the blood pressure monitor 1400 according to embodiment 2 of the present invention, plural case members 1410 to 1440 that are connected by the torque hinge 1460 and the rotation hinge 1450 are included, and the display is provided on the first case member 1410 among the plural case members 1410 to 1440, thereby achieving miniaturization of the device and enhancement of visibility of the screen.

According to the blood pressure monitor 1400 according to embodiment 2 of the present invention, plural case members 1410 to 1440 surrounding the circumference of the cuff 1402 are provided, the plural case members 1410 to 1440 are rotatably connected by the torque hinges 1460 and 1450, and the plural case members 1410 to 1440 can be transformed between the open state in which the cuff 1402 is opened and the stowed state in which the cuff 1402 is enclosed. Additionally, the first and the second locking mechanisms are included to maintain the state in which the cuff 1402 is opened and the state in which the cuff 1402 is enclosed.

Therefore, miniaturization of the device is achieved, and a stable shape can be maintained during use, and particularly miniaturization of the device can be achieved in the stowed state. Furthermore, since the device is compact, flat and rectangular in the stowed state, the storability is enhanced when accumulating plural blood pressure monitors 1400, and it is effective for storing and conveying the monitors in storage, etc.

As explained above, the blood pressure monitor according to the present invention is useful for a sphygmomanometer applied to a subject to measure blood pressure, particularly suitable to a sphygmomanometer that can be transformed between an open state in which a cuff is opened and a stowed state in which the cuff is enclosed therein.

What is claimed is:

1. A blood pressure monitor housing a cuff therein, wherein
at least four case members are rotatably connected with one another in a ring-shape to form a housing unit that houses the cuff, and
the case members rotate to be transformed between a closed state in which the case members are collapsed with the cuff enclosed therein, and an open state.

2. The blood pressure monitor according to claim 1, wherein at least one of the case members includes a display.

3. The blood pressure monitor according to claim 1, wherein an elastic body that exerts a force in a direction that opens the cover is provided on the main body.

4. The blood pressure monitor according to claim 2, wherein an adjusting mechanism that adjusts the force exerted by the elastic body is provided.

5. The blood pressure monitor according to claim 1, wherein the cuff includes a cuff ring, a blood-flow restricting bag outside the cuff ring, and a cuff band outside the blood-flow restricting bag.

6. The blood pressure monitor according to claim 5, wherein the cuff band has a circumferential extension rate lower than an axial extension rate.

7. The blood pressure monitor according to claim 5, wherein
the cuff ring includes two openings through which support members are respectively provided, and
the support members are fitted to the main body and/or the cover.

8. The blood pressure monitor according to claim 1, further comprising a first locking mechanism that includes
a first locking member that is provided on one of the case members that are adjacent in a state in which the cover is open, and
a first hook that is provided on the other of the case members that are adjacent in the state in which the cover is open, and at which the first locking member locks, wherein
the first locking mechanism maintains the state in which the cover is open.

9. The blood pressure monitor according to claim 8, wherein the first locking mechanism includes a first button that unlocks the first locking mechanism.

10. The blood pressure monitor according to claim 1, further comprising a second locking mechanism that includes
a second locking member that is provided on one of the case members that oppose each other in a state in which the cover is closed,
a second hook that is provided on the other of the case members that oppose each other in the state in which the cover is closed, wherein
the second locking mechanism maintains the state in which the cover is closed.

11. The blood pressure monitor according to claim 10, wherein the second locking mechanism includes a second button that connects to and unlocks the second locking member.

12. A blood pressure monitor, comprising:
a cuff;
a plurality of case members that surround an outer circumference of the cuff;
a plurality of hinges that rotatably connects the case members with one another to transform the case members between a open state in which the cuff is open and a stowed state in which the cuff is enclosed; and
a lock member that locks a part of the case members to maintain the open state and the stowed state.
